(19) United States
(12) Patent Application Publication
(10) Pub. No.: US 2020/0143932 A1
(43) Pub. Date: May 7, 2020

(54) SYSTEM AND METHOD FOR BEHAVIOR CHANGE FOR HEALTH AND WELLNESS

(71) Applicant: SQUARE2 SYSTEMS, INC., Hanover, NH (US)

(72) Inventor: Michael John Grabinski, Hanover, NH (US)

(21) Appl. No.: 16/610,755

(22) PCT Filed: May 4, 2018
(86) PCT No.: PCT/US2018/031119

§ 371 Date: Nov. 4, 2019

Related U.S. Application Data
(60) Provisional application No. 62/501,871, filed on May 5, 2017.
(60) Provisional application No. 62/526,468, filed on Jun. 29, 2017.

(51) Int. Cl. G16H 20/70; G16H 50/30; G16H 40/63

(52) U.S. Cl. CPC G16H 20/70; G16H 40/63; G16H 50/30

(57) ABSTRACT

A software application for assisting a user to achieve a behavioral modification goal includes a user interface defining a ladder including a plurality of rungs, each of the rungs having one or more associated tasks, completion of the one or more associated tasks by the user causing the associated rung to be indicated as completed.

### CROSS-REFERENCE TO RELATED APPLICATIONS

This application claims priority under 35 U.S.C. § 119(e) to U.S. Provisional Patent Application Serial No. 62/501,871, titled “SYSTEM AND METHOD FOR BEHAVIOR CHANGE FOR HEALTH AND WELLNESS,” filed on May 5, 2017 and to U.S. Provisional Patent Application Serial No. 62/526,468, titled “SYSTEM AND METHOD FOR BEHAVIOR CHANGE FOR HEALTH AND WELLNESS,” filed on Jun. 29, 2017, each of which being incorporated in its entirety herein for all purposes.

### BACKGROUND

### 1. Field of Invention

The present invention relates generally to systems and methods to assist persons in achieving personal goals. More specifically, systems and methods disclosed herein may provide assistance for a person seeking to alter an aspect of their behavior.

### 2. Discussion of Related Art

When persons identify a behavioral problem or a life goal that they wish to achieve, they may seek the assistance of a therapist or other individual trained in techniques to affect behavioral change. Such techniques may be based in, for example, cognitive behavioral therapy, behavioral activation, problem solving therapy, the community reinforcement approach, or behavioral economics. Therapists are typically trained in a single type of behavior modification therapy. Particular types of behavior modification therapy have been traditionally been recognized as appropriate to address specific types of behavioral change goals.

### SUMMARY OF INVENTION

According to one aspect of the present invention there is provided a software application for assisting a user to achieve a behavioral modification goal. The application comprises a user interface defining a ladder including a plurality of rungs. Each of the rungs has one or more associated tasks. Completion of the one or more associated tasks by the user causes the associated rung to be indicated as completed.

In some embodiments, a first rung must be completed prior to a second rung being unlocked.

In some embodiments, the software application further comprises a ladder selection screen providing for the user to select a ladder designed to address behavior associated with a particular type of issue that the user exhibits.

In some embodiments, ladders designed to address behavior associated with particular types of issues include predefined rungs and associated tasks.

In some embodiments, the ladder selection screen includes an option for the user to create a personalized ladder.

In some embodiments, the software application further comprises a user interface summarizing scores of a plurality of measures associated with different goals.

In some embodiments, the software application further comprises an algorithm that identifies positive or negative trend reversals in measures and directs the user to an appropriate intervention based on a direction of the trend reversal, measure data and other user data.

In some embodiments, the software application further comprises a user interface that displays real time graphing of scores of at least one of the plurality of measures.

In some embodiments, the scores of the plurality of measures include scores related to one or more of fear, anxiety, or discomfort experienced by the user.

In some embodiments, the user interface summarizes the scores of the plurality of measures in a grid.

In some embodiments, a heat map is applied to the scores of the plurality of measures, portions of the grid including individual scores of the plurality of measures being colored in accordance with proximity of respective scores of the plurality of measures to associated target scores.

In some embodiments, colors assigned to the scores of the plurality of measures include white and black to represent extremes of scales of respective measures and shades of a base color to represent intermediate scores of the plurality of measures.

In some embodiments, the shades of the base color are formed by mixing in one of white or black into the base color depending on proximity of the respective scores to the extremes of the scales of the associated measures.

In some embodiments, portions of the grid displaying scores of different measures are colored differently.

In some embodiments, the user interface further comprises an axis including dates associated with respective scores of the plurality of measures.

In some embodiments, the software application further comprises a user interface illustrating a history of scores of the plurality of measures.

In some embodiments, the software application is implemented on a smart phone.

In some embodiments, the software application includes functionality for tracking performance of a repeatedly performed activity.

In some embodiments, the software application includes a user interface which provides for the user to define the repeatedly performed activity and a threshold number of times the repeatedly performed activity is to be performed within a user defined time interval.

In some embodiments, the software application initiates a rescue intervention responsive to the user failing to perform the repeatedly performed activity the threshold number of times within the user defined time interval.

In some embodiments, the rescue intervention includes a questionnaire configured to assist the user in identifying a reason for failing to perform the repeatedly performed activity the threshold number of times within the user defined time interval.

In some embodiments, the software application initiates a reward intervention responsive to the user performing the repeatedly performed activity for more than a threshold number of consecutive time intervals.

In some embodiments, the threshold number of consecutive time intervals is defined by the user.

In some embodiments, the software application further comprises a user interface including one or more features for assisting the user in defining one or more outcome goals.

In some embodiments, achievement of the one or more outcome goals is included in the one or more tasks associated with one or more of the rungs.

In some embodiments, the software application is configured to trigger an event based on data from a measure assigned to an object including one of an exercise and a ladder rung.

In some embodiments, the software application is configured to terminate a requirement for performance of an exercise when a most recent score of a measure associated with the exercise exhibits a defined percent reduction threshold from a peak score for a session of performance of the exercise.

In some embodiments, the software application is configured to unlock a rung on the ladder responsive to a most recent score of the measure exceeding a defined percent reduction threshold from the peak score across all sessions of performance of the exercise.

In accordance with another aspect, there is provided a software application that integrates complementary, science-based techniques for behavior change into one mobile platform.

In some embodiments, the complementary, science-based techniques for behavior change include one or more of cognitive behavioral therapy, behavioral activation, problem solving therapy, the community reinforcement approach to behavior therapy, motivational enhancement therapy, behavioral economics, and future orientation therapy.

In accordance with another aspect, there is provided a software application that integrates validated, science-based techniques to address behavioral problems including one or more of substance use disorders, depression, anxiety, panic disorder, chronic pain, obesity, and binge eating as well as general wellness across ladders within one mobile platform.

In accordance with another aspect, there is provided a software application that integrates validated, science-based techniques that can infinitely scale to include ladders addressing a full spectrum of behavioral health ranging from preventative health to chronic disease management.

In some embodiments, the software application includes functionality to utilize motivational interviewing techniques to re-engage the user after a period of non-use of the software application.

In some embodiments, the software application is configured to initiate notification of the user via one or more of SMS, email, or device notification after an arbitrary period of non-use of the software application.

In some embodiments, activation of the notification by the user opens the software application and engages the user in a process to motivate the user to continue to use the software application.

In some embodiments, engaging the user in the process to motivate the user to continue to use the software application includes using motivational interviewing techniques to resolve ambivalence to continue using the software application.

In some embodiments, responsive to the software application being opened, the software application uses a question and answer process to assess whether the user has relapsed into a target behavior.

In some embodiments, responsive to making a determination that the user has relapsed in to the target behavior, the software application offers a rescue intervention.

In some embodiments, the software application is configured to display a map of triggers for a target behavior.

In some embodiments, the software application is further configured to use a question and answer process to identify triggers for the target behavior.

In some embodiments, the software application is further configured to allow users to add their own triggers via one of free-text input and voice recognition and to identify a category of an added trigger based on where in the question and answer process the trigger was added.

In some embodiments, the software application is further configured to provide a list of commonly known triggers for the target behavior.

In some embodiments, the software application is further configured to allow users to add their own triggers via one of free-text input and voice recognition.

In some embodiments, the software application is further configured to allow users to select from a list of triggers they previously identified.

In some embodiments, the software application is further configured to present a list of alternative behaviors to the user based on one of the selected trigger and a category of the selected trigger, the alternative behaviors selected based on an expected efficacy of the alternative behaviors in alleviating an undesirable response of a user to the one of the selected trigger and category of the selected trigger.

In some embodiments, the software application is further configured to offer alternative responses for triggers based on the trigger itself.

In some embodiments, the alternative responses are selected at least in part based on text of the trigger.

In some embodiments, the software application is further configured to use language stemming to match added trigger to known triggers.

In some embodiments, the software application is further configured to use natural language processing (NLP) to identify a category of an added trigger.

In some embodiments, the category of the added trigger is selected from the group consisting of a person, a place, a time, a thing, a thought, and a feeling.

In accordance with another aspect, there is provided a device configured to execute the software application of any of the aspects or embodiments disclosed above.

In accordance with another aspect, there is provided a device that facilitates the definition of a behavior modification goal by a user, applies behavioral modification techniques to assist the user in achievement of the behavior modification goal, and tracks progress toward achievement of the behavior modification goal by the user.

In some embodiments, the device includes a user interface that provides the user with information regarding progress toward achievement of the behavior modification goal.

In some embodiments, the device includes programming configured to cause the device to display an interface including an entry field for the user to identify a behavior or feeling that the user engaged in or experienced.

In some embodiments, the device includes programming configured to cause the device to display a selection interface providing the user with a list of user-selectable behaviors or feelings that the user engaged in or experienced.

In some embodiments, the programming is further configured to display user-selectable lists of one or both of external or internal cues associated with a behavior or feeling selected by the user in the first selection interface responsive to selection of the behavior or feeling by the user in the selection interface or responsive to the user identifying the behavior or feeling in the entry field.

In some embodiments, the programming is further configured to prompt the user to enter one or both of an external or internal cue associated with a behavior or feeling selected by the user in the selection interface in a free-form text entry field responsive to selection of the behavior or feeling by the user in the first selection interface or responsive to the user identifying the behavior or feeling in the entry field.

In some embodiments, the programming is further configured to display a list of the external or internal cues and, responsive to the user selecting a one of the external or internal cues from the list, to display a list of suggested responses the user may undertake to mitigate the behavior or feeling.

In some embodiments, the programming is further configured to display a list of the external or internal cues in order of frequency of occurrence.

In some embodiments, the programming is further configured to guide the user through performance of a response that the user selects from the list of suggested responses.

In some embodiments, the device includes programming configured to assist the user in defining process goals that when performed, progresses the user toward achieving the behavior modification goal.

In some embodiments, the programming is further configured to record and display instances of the user performing a task involved in achieving a process goal.

In some embodiments, the programming is further configured to trigger a reward intervention for the user responsive to the user performing the task a predetermined number of times without missing a threshold criteria of instances of performing the task.

### BRIEF DESCRIPTION OF DRAWINGS

Various aspects of at least one embodiment are discussed below with reference to the accompanying drawings. In the drawings, which are not intended to be drawn to scale, each identical or nearly identical component that is illustrated in various drawings is represented by a like numeral. For purposes of clarity, not every component may be labeled in every drawing. The drawings are provided for the purposes of illustration and explanation, and are not intended as a definition of the limits of the invention. In the drawings:

FIG. 1 is an example of a welcome screen for a behavioral modification assistance app;

FIG. 2 is an example of a Grid Summary screen;

FIG. 3 is an example of a Grid Detail screen;

FIG. 4 is an example of an event record entry screen;

FIG. 5 is an example of an event record summary screen;

FIG. 6 is an example of a Measure definition entry screen;

FIG. 7 is an example of a Chain definition entry screen;

FIG. 8 is an example of a Thinkspace screen;

FIG. 9 is an example of a Plan Workspace Screen;

FIG. 10 is an example of a List Workspace screen;

FIG. 11 is an example of a Pros/Cons Workspace screen;

FIG. 12 is an example of a Ladder selection screen;

FIG. 13 is an example of a Ladder rung preview screen;

FIG. 14 is an example of a Ladder that a user may use to help with panic attacks;

FIG. 15 is an example of a Ladder that might be used by someone with agoraphobia;

FIG. 16 is an example of a chart of Measure values over time;

FIG. 17 is a flowchart of a method that may be performed utilizing examples of the system and app disclosed herein;

FIG. 18 is another example of a Grid Summary screen;

FIG. 19 is an example of a list of triggers for an undesired reaction, feeling, or behavior;

FIG. 20 is an example of an interface for adding responses to address a trigger;

FIG. 21 is an example of a list of suggested responses to address a trigger;

FIG. 22 is an example of a screen displayed responsive to selection of a response in the list of suggested responses in FIG. 21;

FIG. 23 is an example of a list including a single response to address a trigger;

FIG. 24 is an example of a screen displayed responsive to selection of the response in FIG. 23;

FIG. 25 is an example of a screen providing instructions for a user to perform a selected response to a trigger; and

FIGS. 26-32 illustrate examples of interfaces utilized by the application to help a user identify triggers for undesirable reactions, feelings, or behaviors.

### DETAILED DESCRIPTION

Behavioral health issues are prevalent in modern society. For example, it has been estimated that approximately 10% of the population suffers from some form of substance abuse and that about 25% of the population suffers from some form of mental health issue. It has also been estimated that about 40% of poor outcomes in healthcare can be attributed to poor behavior, for example, smoking, eating poorly, or exercising insufficiently or not at all. Yet approximately 90% of people who suffer from behavioral health issues fail to obtain treatment. This large percentage of people who fail to seek treatment may be due to multiple causes, for example, the inconvenience of obtaining treatment, the cost of obtaining treatment, or fear of the stigma of being labelled as someone who is mentally ill.

To make behavioral therapy more accessible to people, aspects and embodiments disclosed herein present a system and method that may be implemented on a personal computer or portable computing device, for example, a smart phone. A person may have constant or nearly constant access to a guide for performing behavioral modification exercises to address desired goals or to reduce or eliminate the occurrence of unwanted behaviors without having to visit a therapist and that the person can use privately, eliminating some of the barriers that prevent many people from seeking behavioral modification therapy. A user may utilize the system and method to address behavioral issues in real time, without having to wait for a therapist appointment.

It has been discovered that many forms of behavioral modification therapy rely on a set of common techniques or “active ingredients.” Consistent with the techniques for behavior change listed above, these “active ingredients” include helping motivate individuals to identify personal goals, identify barriers to achieving goals, and learn new skills and strategies to initiate and maintain goal-directed behavior change These “active ingredients,” which are embedded within specific forms of behavior modification, may be generalized to address a wide range of behavioral modification goals, even to goals that particular techniques have not been traditionally applied. Generalization of these different techniques may provide an opportunity to transcend siloed therapy methodologies or mobile health apps that focus on a single disorder to embrace integrated care for a full spectrum of behavioral modification goals. Various aspects and embodiments disclosed herein offer science-based self-regulation monitoring and health behavior change tools via an integrated platform to a wide array of populations. Various aspects and embodiments disclosed herein provide a mobile platform that employs the fundamental principles behind the science of behavior change to apply to a broad array of populations based on their goals, needs, and preferences and independent of diagnosis, disease, or disorder.

The techniques employed by various systems and methods disclosed herein include validated, science-based techniques to address a wide range of behavioral problems, including substance abuse, depression and anxiety, as well as chronic physical health issues with behavioral components, such as diabetes, heart disease and obesity. The techniques and skills are not specific to those for use in mental health treatment. They can just as easily be applied to any goal, including weight loss, smoking cessation, fitness, emotional well-being, and academic and professional achievement.

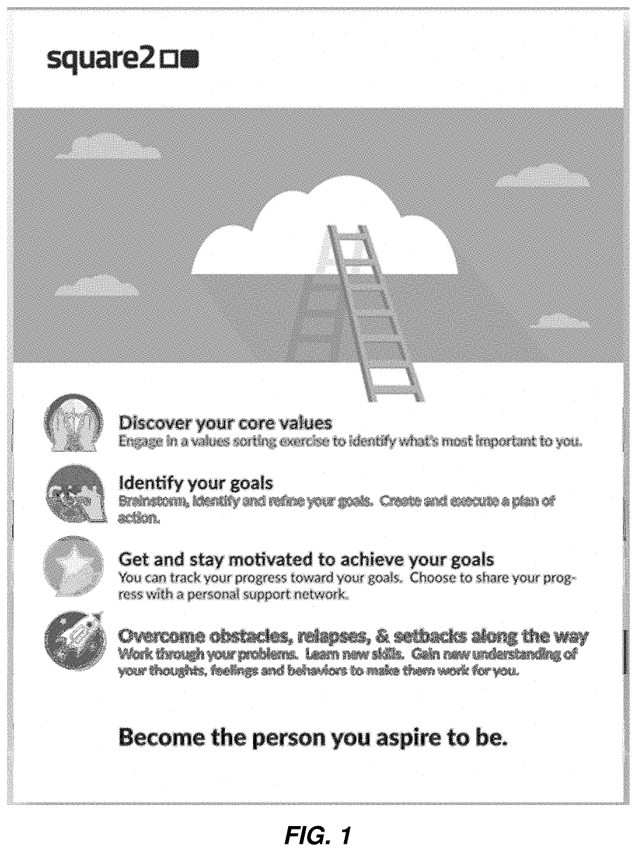

Aspects and embodiments disclosed herein may be implemented as an app on a smart phone. The app may include a number of core tools. “Guides” teach valuable skills and provide expert advice for tackling common problems. Guides walk a user through well-researched techniques for achieving goals and overcoming setbacks and obstacles. Unfinished guides and those that a user has added to a reading list may appear under a Bookmarked tab in the Guides section of the app so users can pick up where they left off. Access to Core Guides may be provided on a guide introduction screen, for example, as illustrated in FIG. 1. The Core Guides may assist a person in, for example, identifying their core values, identifying their goals, and setting their goals.

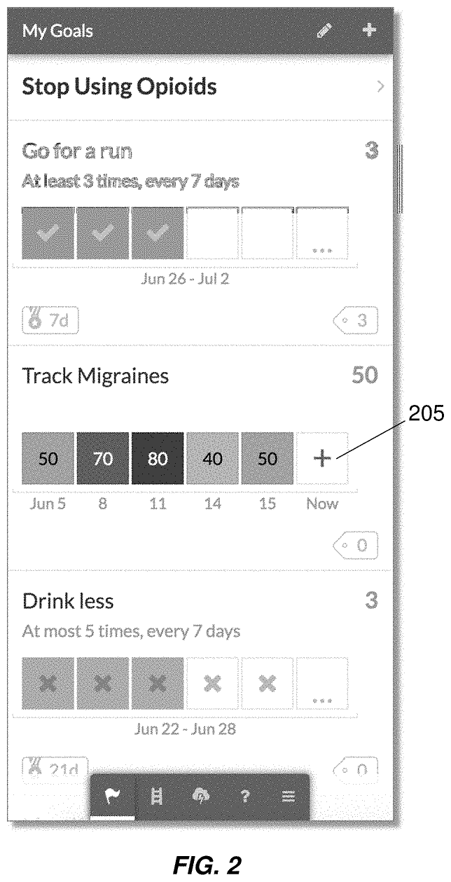

Users track progress towards their goals on a “Grid.” An example of the Grid is illustrated in FIG. 2. A user may enter progress on the Grid for the current and past days. The date range made visible on the grid can be changed by swiping from the left of the screen to move backwards in time and swiping from the right to move forward in time. A user may see the status on all their goals on a Grid Summary screen.

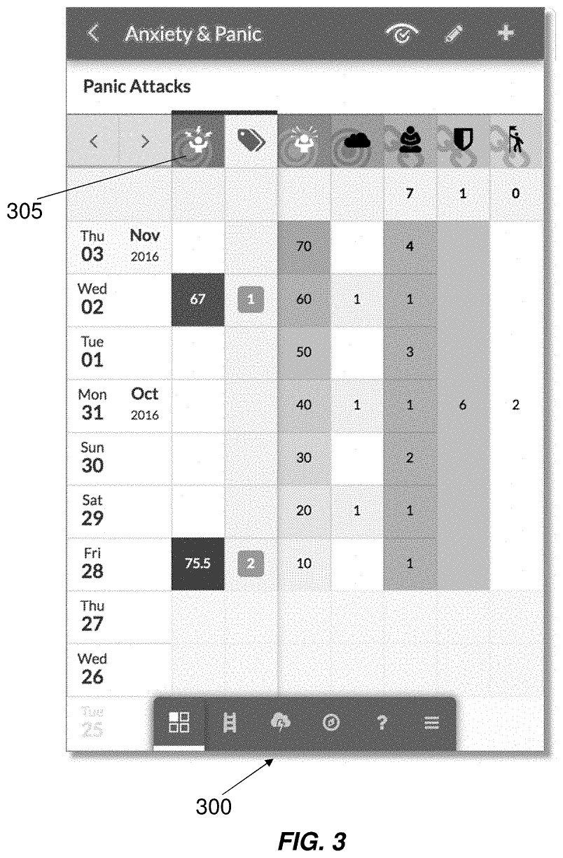

Clicking on a grid or goal name will take the user to a Grid Detail screen where the user can see past progress on all goals in the grid. A user can also back-enter past progress on the Grid Detail screen. An example of a Grid Detail screen is illustrated in FIG. 3. The various icons at the top of the columns in the Grid Detail screen identify the columns as providing historical data for the measure having the same icon in the Grid Summary screen. The items in the column between the column having the “Panic Attacks” icon (the third column in the grid of FIG. 3) and the column having the “Anxiety and Worry” column (the fifth column in the grid of FIG. 3) includes links (squares labelled “1” and “2”) to access results from exercises performed to help alleviate the negative experiences. The Grid Detail screen may apply a heat map to different measure to indicate measures that are considered to be at desirable or undesirable levels.

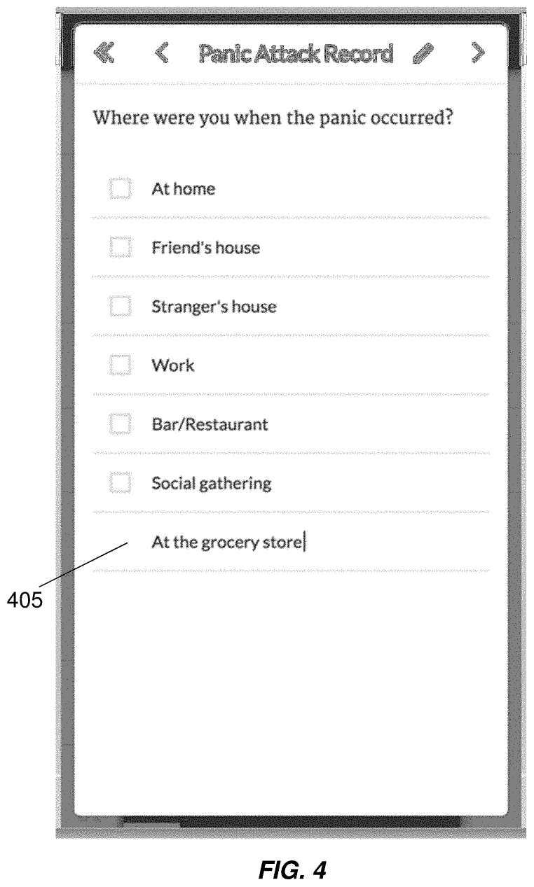

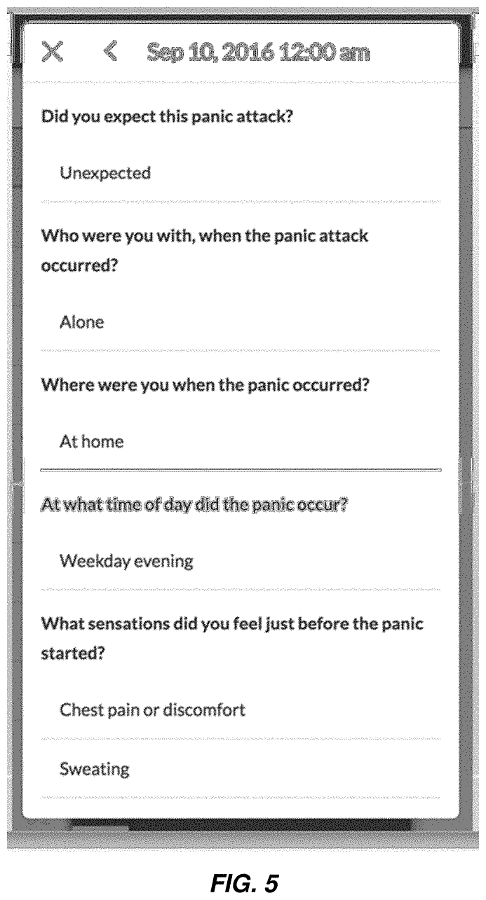

Adding a new record to a Grid may be performed by selecting an icon representing the type of record desired in the Grid Summary or Grid Detail screen. For example, if a user experiences a migraine, they may select the plus sign icon 205 on the grid for “Track Migraines” in the Grid Summary screen illustrated in FIG. 2. Selecting the plus sign icon may trigger a migraine record where the user can log the antecedent events that may have precipitated the migraine. Similarly, on the Grid Detail screen illustrated in FIG. 3 the selection of the panic attack face icon 305 may cause a panic attack record entry screen or series of screens to open in which the user may enter the details of the panic attack. FIG. 4 illustrates a first in a series of panic attack record entry screens. Record entry screens such as that illustrated in FIG. 4 may include a list of common cues or triggers for a feeling or behavior the user have experienced or performed from which the user may select, and may also include a free-form text entry field (indicated at 405 in FIG. 4) in which the user may enter a new cue or trigger that is not on the pre-populated list, for example, by typing or by utilizing speech-to-text voice recognition functionality of a device upon which embodiments of the software app disclosed herein may be installed. A number of screens may be presented to collect information about the panic attack that may be then summarized in an event record summary screen as illustrated in FIG. 5. The data entered may provide for the system to assign a severity score to the panic attack, for example, the severity scores of 75.5 for the panic attack that occurred on October 28 and the severity score of 67 for the panic attack that occurred on November 2, shown in the Grid Detail screen illustrated in FIG. 3. Alternatively, the user may enter a self-assessment of the severity of the panic attack, for example, in one of the of panic attack record entry screens, that may be used as the severity score.

The navigation buttons on the bottom of the Grid Summary or Grid Detail screens (indicated generally at 300 in FIG. 3), and other screens displayed by the app may be used to access different functionality or different information on the app. The function of the navigation buttons, from left to right may be, for example, access Grid Summary screen, access Ladder summary screen (described below), access Workspaces (described below), access Guides, access help functions specific to a particular screen being displayed, and access app functions (settings, logout, etc.).

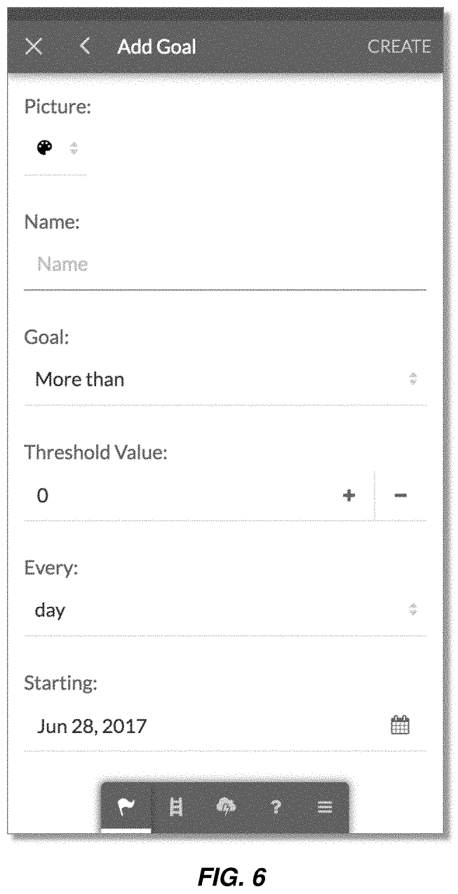

When defining a goal, the app provides for a user to define Measures. Measures define what a person wishes to accomplish and may be considered outcome goals. Measures may include anything associated with a goal that one can count or quantify. Different Measures may include, for example, weight, hours of exercise, amount of weight one can bench press, time spent studying, completed pages of a novel, cigarettes per day, number of new clients, etc. For Measures with a starting value, an ending value, and an end date (for example, drop from 200 pounds to 180 pounds in three months) the app will calculate a daily target and show it on the Grid Detail screen. Hitting your daily targets insures you will achieve your goal. In some instances the app may use Natural Language Processing (NLP) to recognize phrases of the form “FROM x TO y BY time/date” as Measures, for example, “FROM 200 pounds TO 180 pounds IN 2 months,” “FROM 7 cigarettes per day TO 0 IN 2 weeks,” “FROM running 2 miles TO 26 miles BY June 1st,” and “FROM a 2.0 GPA TO a 3.0 GPA BEFORE next year.” An example of a screen that may be used to enter a Measure is illustrated in FIG. 6.

The system will display a visual measure indicator in any input box where text is entered when it recognizes an entered goal statement as a Measure. By default, clicking on a data entry cell for a Measure on the Grid Summary or Grid Detail screens will result in a universal data entry process being invoked. A Measure can be defined to launch a process that can implement a single or multi-step data-entry process to calculate a final score for the Measure, which is then entered as the value for the cell on the grid.

The app also allows one to create or define “Chains.” Training new behaviors or developing new habits is a key component of achieving most goals. Chains help a user do this. They represent “process goals”—actions a user repeats to help achieve a desired outcome goal.

If a user wanted to lose 20 pounds, for example, the user might set up multiple chains: “Going to the gym 3 times a week,” “Eating less than 1800 calories per day,” etc.

One goal when utilizing the app is to keep a chain intact for as long as possible. By not breaking the chain, a user may create a new habit that moves the towards their outcome goal and/or away from an undesired behavior or away from instances of experiencing an undesired feeling.

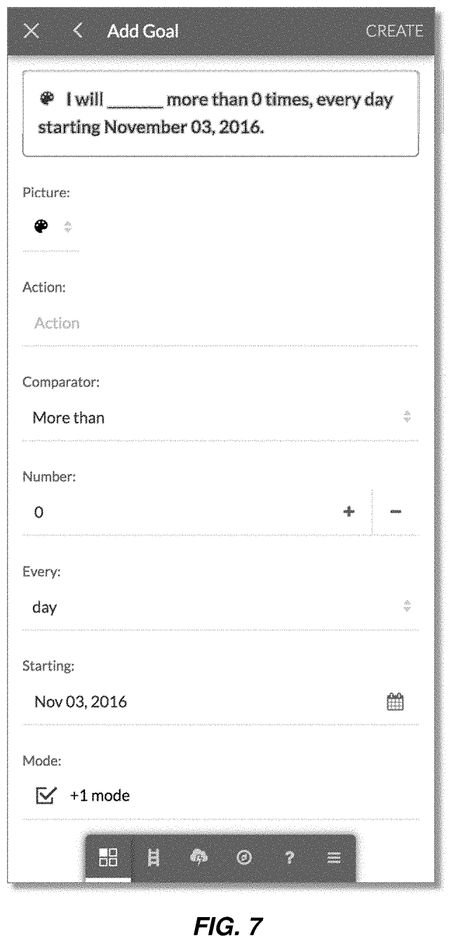

The system may use NLP to recognize statements as Chains when they are entered as “what, how much, how often” phrases and may display a visual Chain indicator in the workspace when an entered statement is recognized by the app as a Chain. The Chain indicator may be displayed in any input box where text is entered. The following are phrased as Chains: “Eat an apple every day,” “Eat at least 1 apple every day,” “Exercise at least 6 times per week,” “Go out with spouse twice every 2 weeks,” “Eat an apple at least twice a day.” An example screen for defining a Chain is illustrated in FIG. 7.

By default, clicking on or otherwise selecting a data entry cell for a Chain on the Grid Summary or Grid Detail screens will result in a 1 being added to the value of the cell. A Chain can be defined to launch a Process on click, however. For example, for a Chain that says you should practice your “Breathing Exercises” 7 times per week, if the user clicks on or otherwise selects the data entry cell, the system will open the breathing exercise, allowing the user to practice. When the user clicks or selects POST they get a +1 added to their chain count for practicing.

The app further defines or allows a user to define Intervention Algorithms which may be instantiated upon the occurrence of different events. The system intervenes differently depending on whether the goal is a Chain or a Measure.

A break in a chain may trigger a “Rescue” intervention. A break occurs when the user fails to meet the threshold criteria for a given interval. For example, if the chain is defined as “Eat an apple at least twice a day” and the user records “1 apple” in a given day then the threshold of 2 apples for the interval of 1 day has not been met and the Chain is broken. A Rescue Intervention is designed to troubleshoot the user's problems and restore the positive trend. A Rescue Intervention may include, for example, a questionnaire to help the user resolve or understand what caused them to break a Chain.

A “Reward” intervention may be triggered at different unit intervals, for example, 1, 7, 14, 21, or 28 unit intervals. The unit is determined by the time interval defined by the Chain. Additionally, for chains with an interval of 1 day the system may define additional trigger points of, for example, 3 months, 6 months, and 1 year. Reward Interventions are designed to reward the user for continuing a positive trend. A Reward Intervention may include, for example, a congratulations message sent to a user's email, or the award of a virtual medal or gold star to the user.

Progress for a Measure is interrupted when there is a negative trend reversal. In some embodiments, a trend reversal is triggered using a Percentage Price Oscillator (PPO) equation calculated on a 3 and 11 sample Exponential Moving Average (EMA). When the PPO transitions from 0 or above to below 0, a Rescue Intervention process is triggered. When the PPO transitions from 0 or below to above 0, a Reward Intervention process is triggered.

In various embodiments, the app may allow a user to create or utilize predefined Workspaces. Workspaces are processes that provide an interface for developing ideas, for example, goal setting and problem solving, and to weigh options when making important decisions. Workspaces can also store the results of the work of a user for later reference.

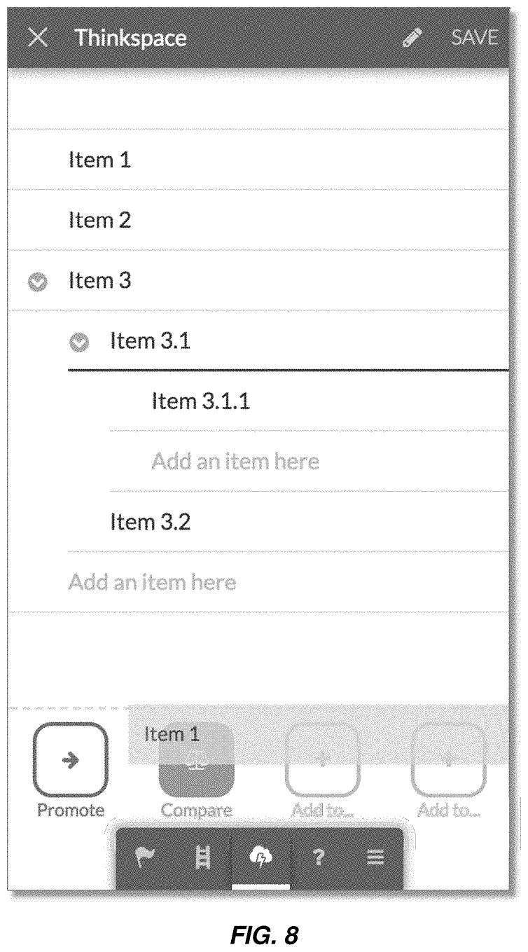

There may be a few basic Workspace types including, for example, Thinkspaces, Plan Workspaces, List Workspaces, and Pros/Cons Workspaces. In Thinkspaces a user may enter in a number of ideas and refine them into focused goals they can act on. An example of a Thinkspace screen is illustrated in FIG. 8. A Thinkspace allows one to create a nested list. One can reorder items in the list by dragging them from their current position to the new position. One can drag and drop goals onto a dropzone, for example, the “Promote” and “Compare” regions on the Thinkspace screen illustrated in FIG. 8, and the action indicated on the dropzone will be performed on the item. For example, dropping an item on the Promote dropzone will send the item to the next step in the Thinkspace process. A Thinkspace is composed of an indefinite number of steps. A step gets added when an item is promoted or compared and the item is added to that new step. Dropping an item on the Compare dropzone sends it to an intermediate Pros/Cons step.

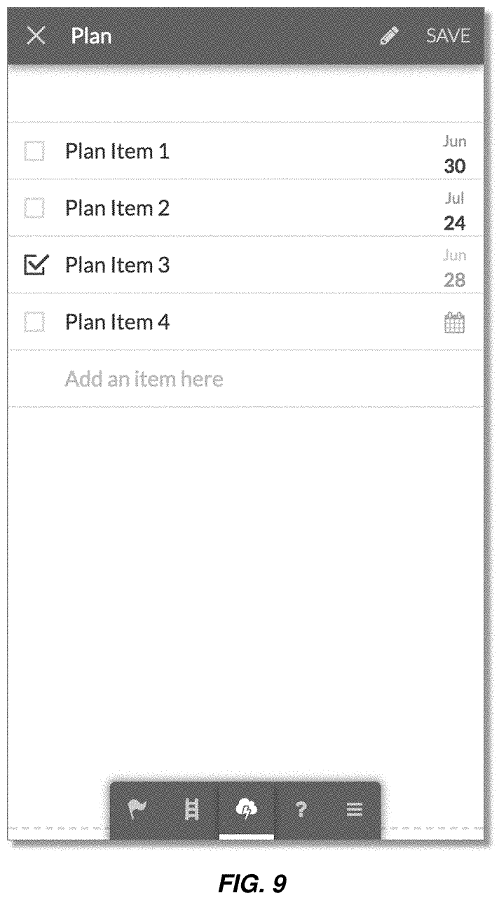

Plan Workspaces provide for a user to lay out a sequence of tasks for meeting a goal or provide a user with at least partially predefined sequences of tasks to help keep on track with regard to maintaining a Chain or achieving a Goal. A Plan is a sequential list of tasks. Each task can be marked completed. Each task may be associated with a deadline. An example of a screen illustrating Plan tasks and deadlines and allowing one to indicate completion of Plan tasks is illustrated in FIG. 9.

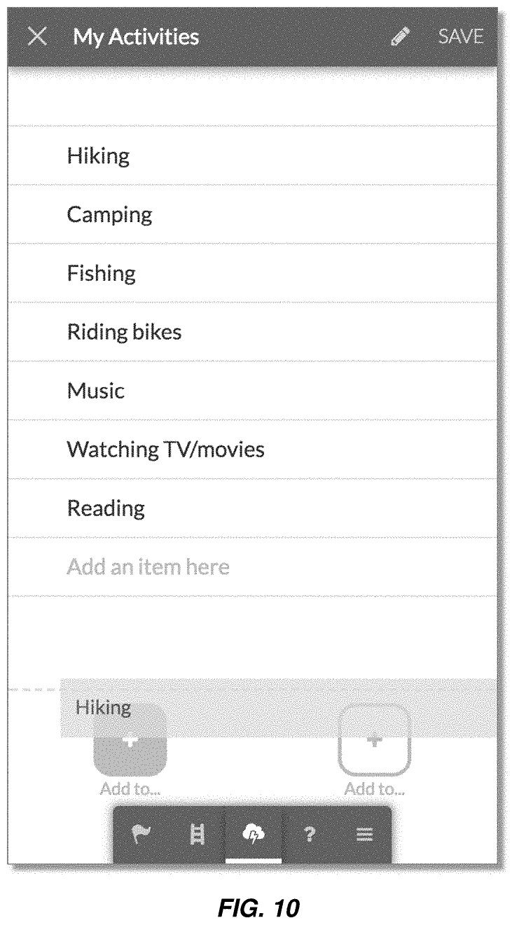

List Workspaces provide for a user to make multi-level lists. An example of a Lists Workspace screen is illustrated in FIG. 10.

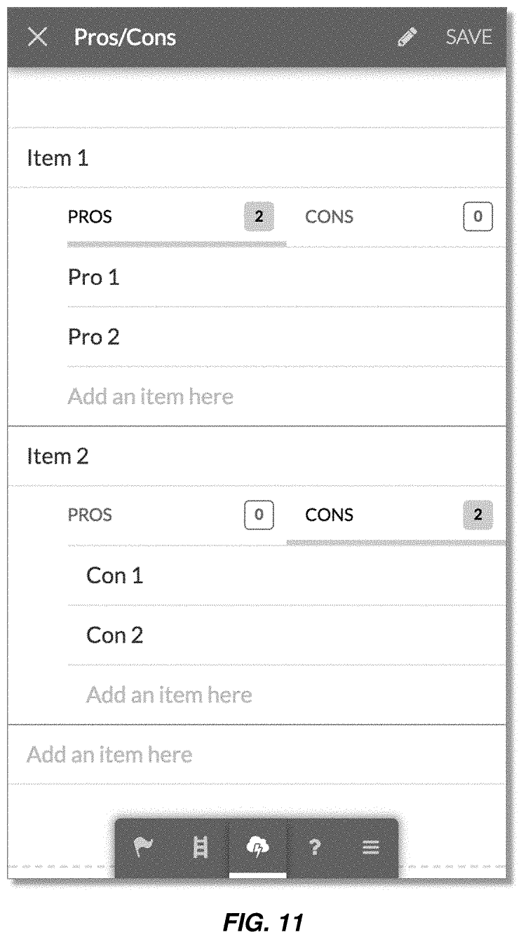

Pros/Cons Workspaces assist a user in choosing the “best” option among many possibilities. Pros/Cons Workspaces provide side-by-side lists to list the benefits (pros) and costs (cons) of an item. An example of a Pros/Cons Workspace screen is illustrated in FIG. 11.

A user can move items from one workspace to another, either by dragging and dropping onto the provided drop targets at the top of each Workspace, or by copying and pasting using the editing tools provided by the app when the user activates edit mode (indicated by the pencil icon in the top-right of the app user interface).

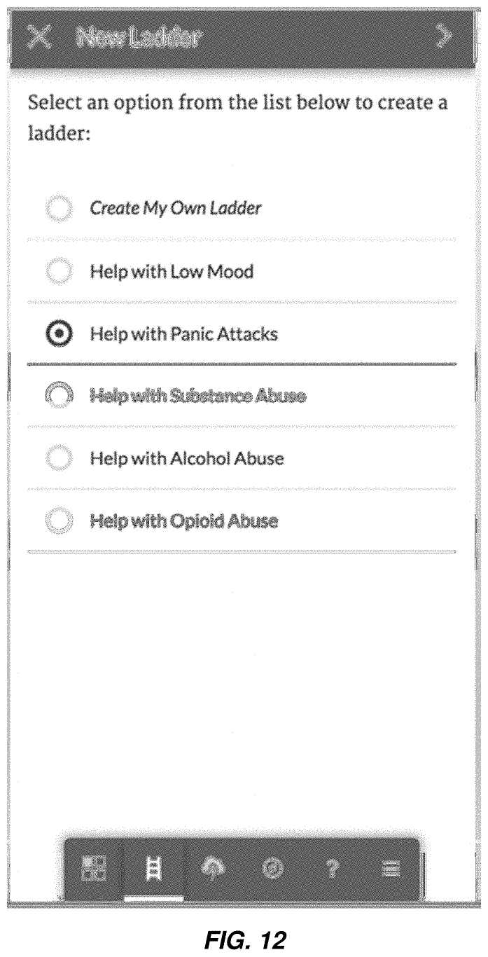

Aspects and embodiments disclosed herein may present a user with tasks to perform to achieve a defined goal in the form of a Ladder. Ladders organize guides, goals and tools into a cohesive package. A ladder is made up of rungs. A rung can contain multiple guides or milestones (items). In some embodiments, initially only the first rung is unlocked. Items on a rung can be accessed in any order. When all items on a rung are completed the next rung in the ladder is unlocked. In some embodiments, the rungs must be unlocked in sequence. In some embodiments disclosed herein the app includes various predefined Ladders to address particular problems. Within predefined Ladders, a user may still have the option of setting or modifying specific goals, tasks, or targets. An example of a ladder selection screen is illustrated in FIG. 12. As illustrated in FIG. 12, in some embodiments a user has an option of either selecting a predefined Ladder or for creating their own customized Ladder to address a problem that is not reflected in one of the predefined Ladders.

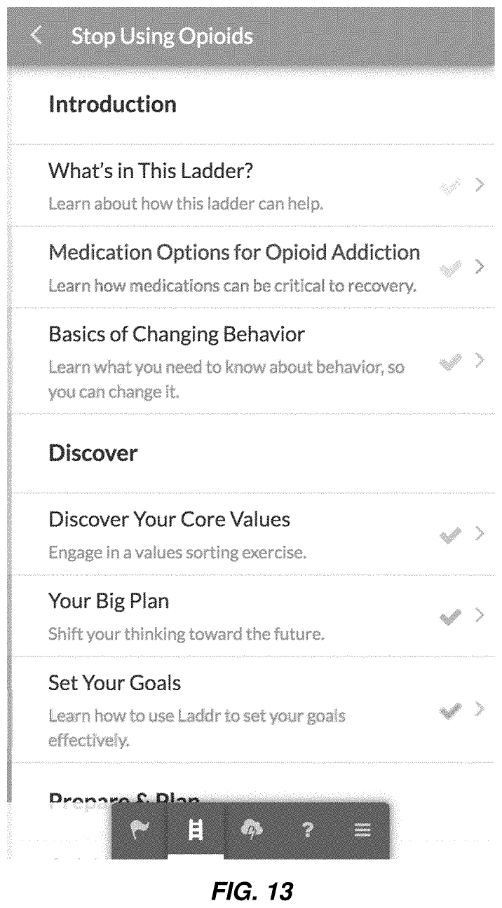

In some embodiments a user may be presented with a preview of all rungs in a Ladder prior to beginning work to complete the rungs of the Ladder. The preview may be accessed even after beginning work to complete the Ladder if desired by a user. An example Ladder preview is illustrated in FIG. 13.

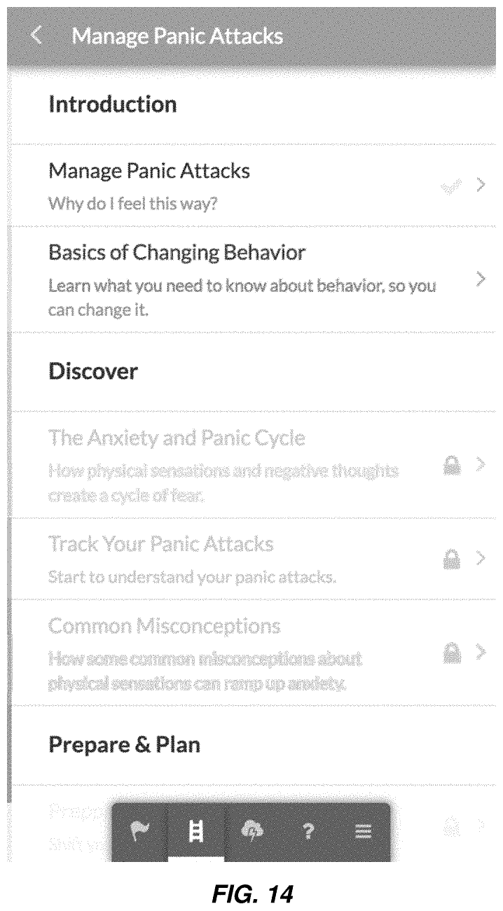

An example of a Ladder that a user may use to help with panic attacks is illustrated in FIG. 14. The rungs are illustrated in a vertical sequence, although in other embodiments, different arrangements of rungs may be utilized. Completed rungs are indicated in the example of FIG. 14 with checkmarks. Rungs that have been unlocked by still include tasks left to complete, for example, the “Basics of Changing Behavior” rung in FIG. 14 is illustrated without a completion checkmark. Rungs that are yet to be unlocked, for example, the “The Anxiety and Panic Cycle” rung in FIG. 14 are greyed out or include a lock symbol.

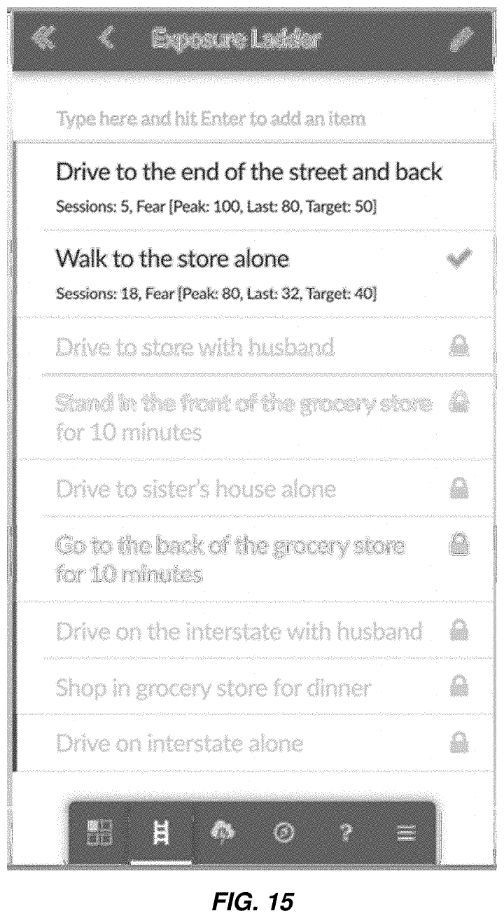

Another example of a Ladder that might be used by someone with agoraphobia, a fear of public places, or a fear of interaction with others is illustrated in FIG. 15. In this Ladder, a first rung includes the task of driving to the end of the street and back. The Ladder illustrated in FIG. 15 indicates that the user has performed this task 5 times (5 “Sessions”), experienced a peak fear score of 100, and a fear score of 80 during the last session. The Ladder illustrated in FIG. 15 also indicates that the first rung may be completed when the user achieves a target fear score of 50 during a session. The second rung “Walk to the store alone” was worked concurrently with the first rung. The second rung was completed after 18 sessions because the user achieved a peak fear score of 32, below the goal of 40, during the last session. The checkmark in the second rung indicates it was completed. Additional rungs in the Ladder remain locked until the first rung is completed.

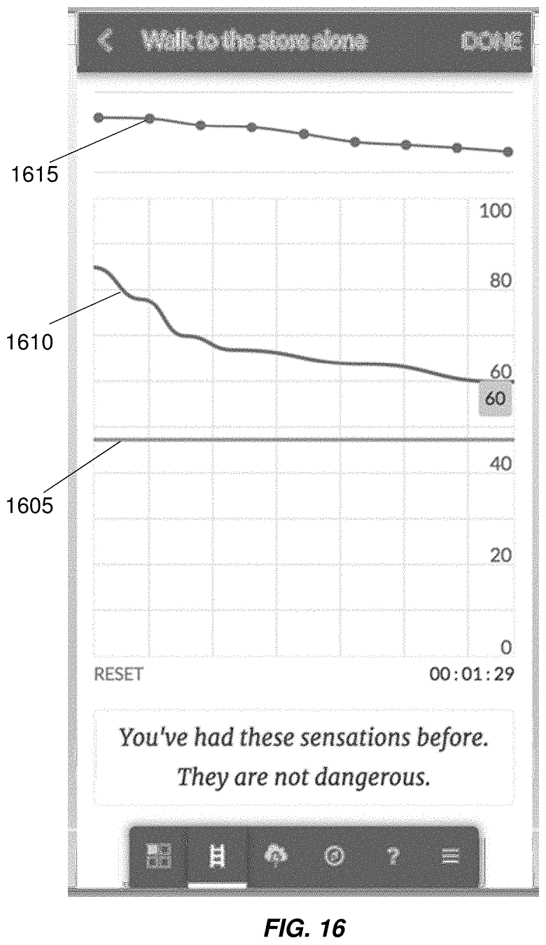

In addition to monitoring one's progress toward achieving a goal or completion of a rung of a Ladder by reviewing a Grid Summary screen such as illustrated in FIG. 2 or Grid Detail screen as illustrated in FIG. 3, in some embodiments, the system may provide a user with a chart of scores for a Measure over time. FIG. 16 illustrates one example of a chart of fear scores over time for the “Walk to the store alone” rung of the ladder of FIG. 15. In FIG. 16 the horizontal line indicated at 1605 may represent a target level for the score of the measure and the solid downwardly trending line indicated at 1610 may represent progress toward the goal for the Measure over a rolling period of time. In some embodiments, the time series score chart may also present a user with a representation of scores for the Measure over a different time period, for example for all recorded scores for the measure, represented by the line with the circular indicators indicated at 1615 in FIG. 16.

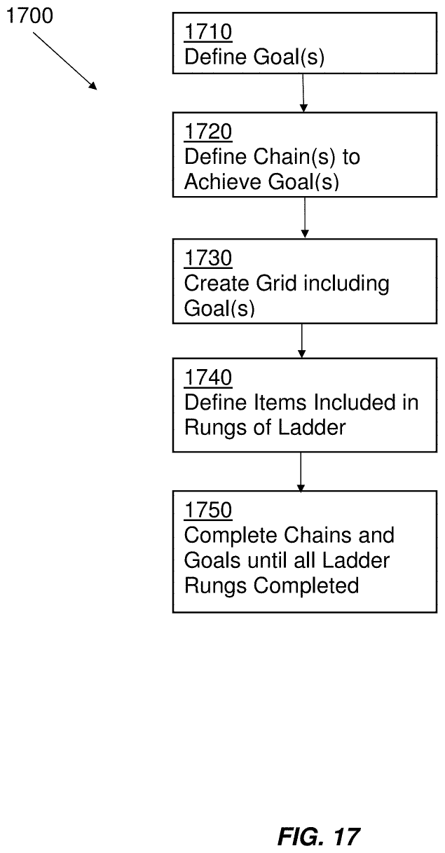

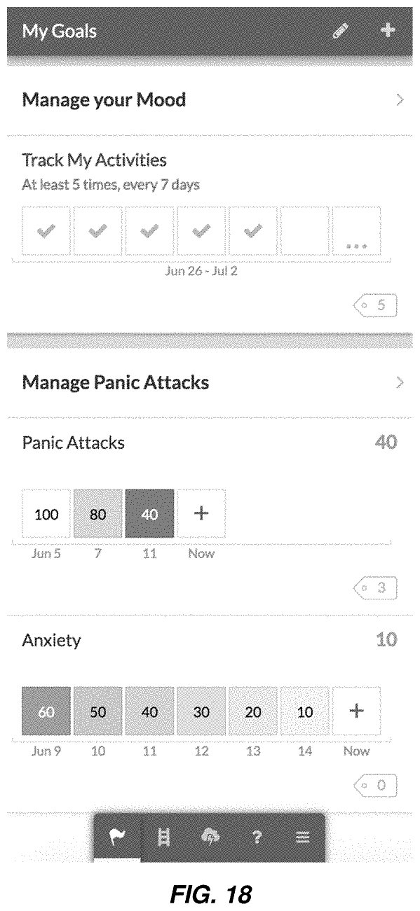

A method in accordance with an embodiment disclosed herein is illustrated in the flowchart of FIG. 17, indicated generally at 1700. This method may be performed on a computer or mobile device, for example, a smartphone in which an app as described herein has been installed or through which such an app may be accessed, for example, over the Internet.

In act 1710, a user identifies a goal that the user wishes to accomplish. The goal may be an outcome-based goal, for example, “I want to lose weight.” The goal may also be an action based goal, for example, a goal that answers the question “What actions must I take to lose weight.” Identifying and setting a goal may involve identifying a specific desired outcome, for example “I want to lose 20 pounds.” The goal should be measurable, including a parameter that can be measured to answer the question “Have I made progress toward achieving my goal?” The goal should be achievable. For example, if the user has never run before, a goal of “I want to run in a marathon in one month” may not be appropriate. It may be appropriate to break more ambitious goals into multiple goals that can be achieved within reasonable time frames. When defining short term goals, these short term goals should be relevant to achieving the more long term ambitious goal. A time frame should also be assigned to a goal. If a goal is expected to take a long time to achieve, it may be appropriate to break the long term goal into multiple shorter term goals. For example, if a user's goal is “I want to lose 50 pounds by next year,” they may want to create sub-goals such as “I want to lose at least 4 pounds per month.”

To enter a user's goal into the app, the user may access a goal setting screen, such as illustrated in FIG. 6 and enter the goal in the form of a Measure. The app may utilize natural language processing to understand Measures that the user enters in a format similar to “FROM x TO y BY time/date,” for example, “FROM 200 pounds to 150 pounds BY January 1 of next year.” The app may provide an indicator in the goal setting screen when it recognizes a Measure entered by a user. The user may enter multiple Measures in the goal setting screen. With Measures a user can track whether their actions are getting the user closer to their ultimate goal and make changes if they are not. The user may enter a long term or ultimate goal and sub-goals beneath the ultimate goal, and possibly sub-sub-goals beneath the sub-goals to create a goal hierarchy. The app may require that all goals in the hierarchy be completed before a rung associated with the goal in a Ladder is marked as completed.

Once a Measure is defined, a user may define a Chain that will help them achieve the Measure. (Act 1720.) A Chain is a behavior, that when repeated may help a user achieve a desired outcome, for example, as defined by a Measure. Many fundamental shifts in behavior require repetition over time, so practice of Chains may be important in reaching a goal. Actions (Chains) such as “I will exercise for 1 hour at least 6 days a week” or “I will dink an 8 oz glass of water at least 8 times a day” may help a user achieve a weight loss goal. An example of a screen for defining a Chain is shown in FIG. 7. The app may recognize statements as Chains when they are entered by a user as “what, how much, how often” phrases. The app may display an indicator in the Chain defining screen when a statement entered by a user is recognized by the app as a Chain.

Once a user has defined a Goal in as much specificity as desired, the user may add the Goal to a Grid. (Act 1730.) Grids help organize Chains and Measures. A user may add all goals on a single Grid or create multiple Grids for multiple long term Goals. A Measure or Chain may be added to a Grid by selecting the “<Add Goal” menu item the Measure or Chain creation screens in FIGS. 6 and 7. Thinkspace, Plan, or List items may be added to a grid by dragging and dropping an item into the “Add to . . . ” drop targets in the screens illustrated in FIGS. 8-10 and then selecting the name of the targeted Grid Adding Goals to a Grid allows for a user to track their progress toward achieving the Goals. Tracking their progress may allow the user to help discover patterns in their behavior, let the app present troubleshooting advice or exercises, and keep the user motivated.

Different Goals or Chains or groups thereof may be added to different rungs of a Ladder. (Act 1740.) Selecting a particular rung on a Ladder may cause a Grid Summary associated with the rung to be displayed. A user may enter progress toward their Goals via the Grid Summary (Act 1750) until all Goals or Chains associated with the rung are completed and the user may move on to a new rung until the entire ladder is completed.

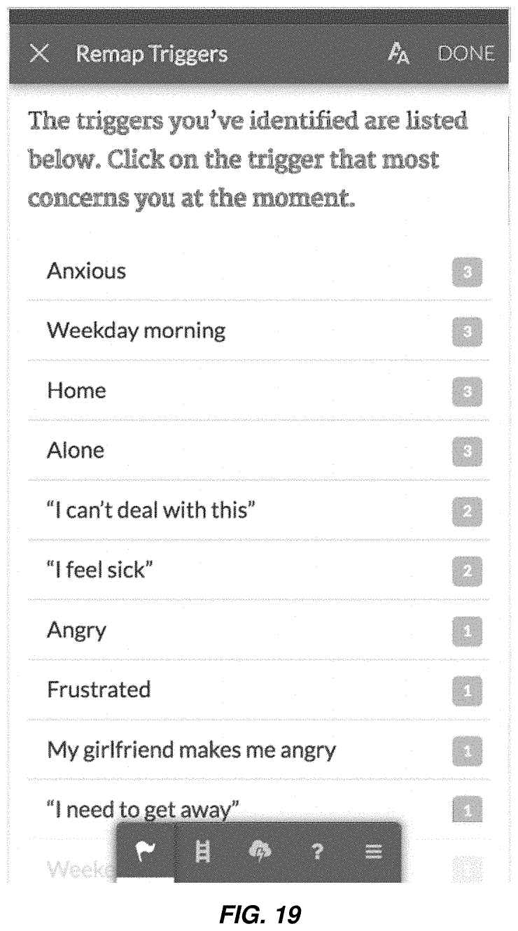

In some embodiments, the application may guide a user in responding to occurrences of an undesirable behavior or feeling. If a user provides the application with an indication of the occurrence of an undesirable behavior or feeling, for example, an occurrence of a substance misuse, the application may provide the user with suggestions on how to mitigate or otherwise deal with the undesirable behavior or feeling. FIG. 19 illustrates a list of triggers (also referred to as “cues” herein) identified by users that may have contributed to or caused the occurrence of the undesirable behavior or feeling. The list is ordered by the number of occurrences for a trigger. The more frequently occurring triggers are at the top of the list. The user may select a trigger from the list. In other embodiments, the triggers may be displayed in bubble plots of triggers by number of occurrence or word clouds of triggers by number of occurrence.

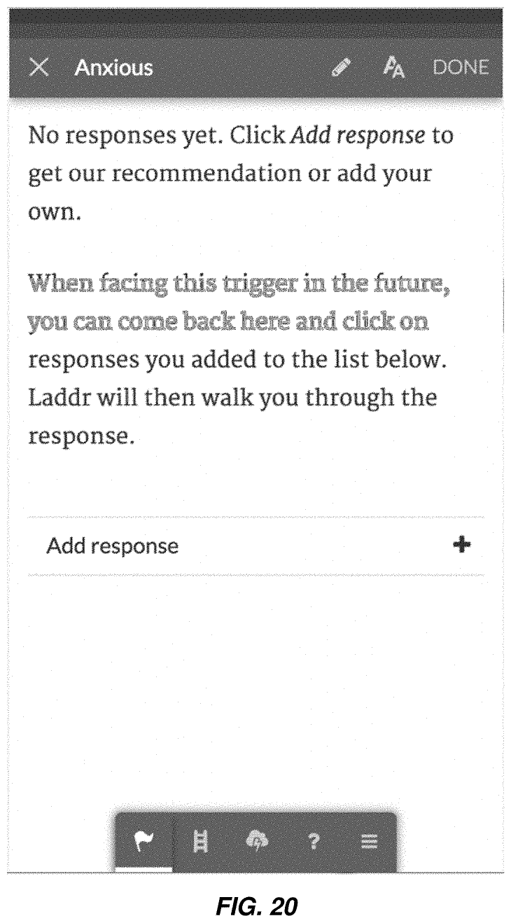

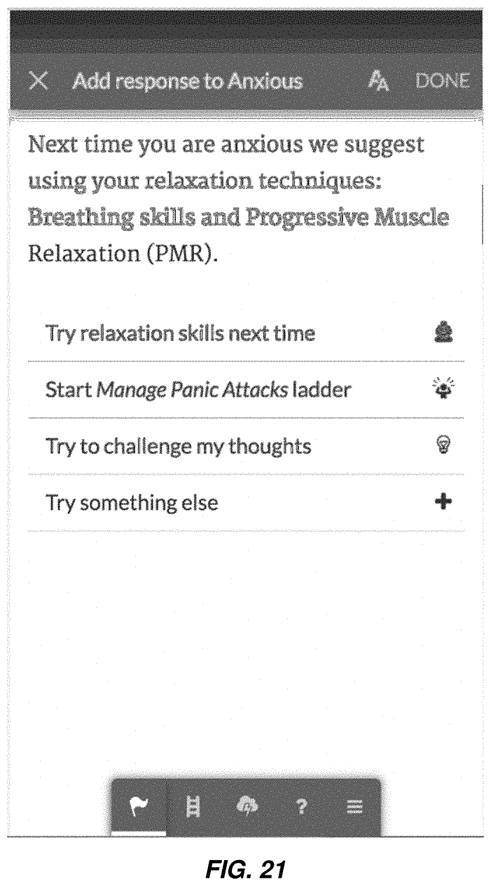

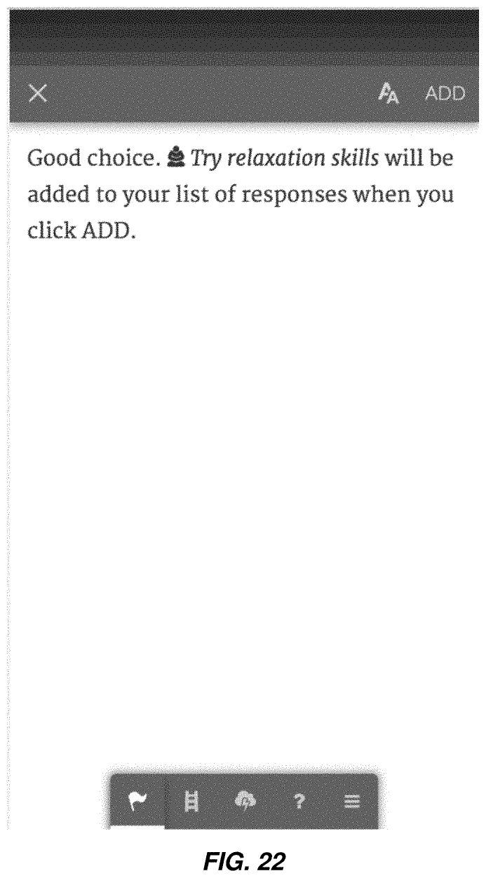

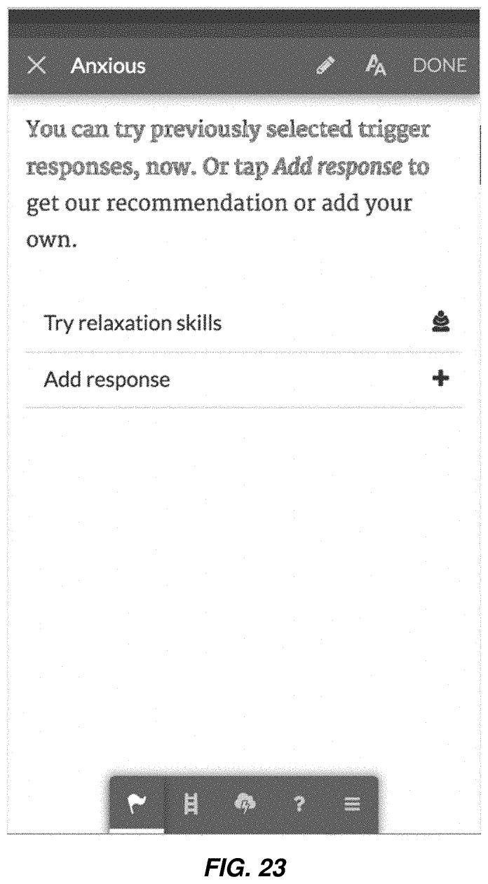

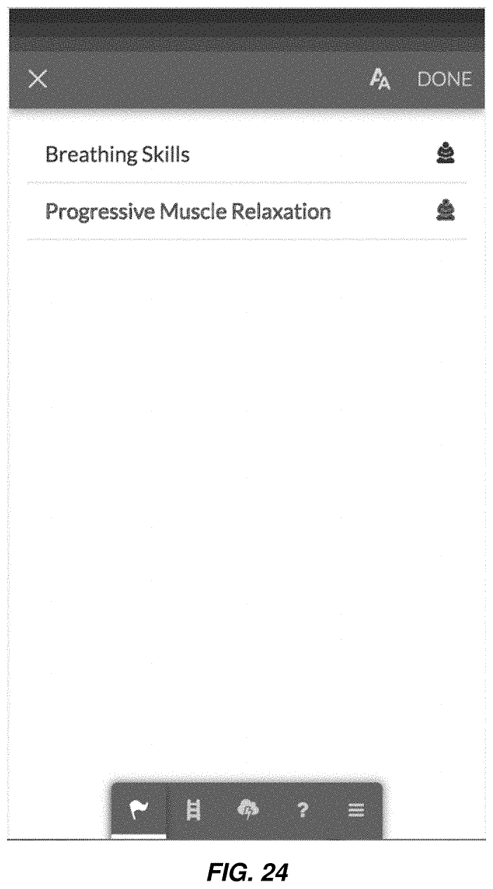

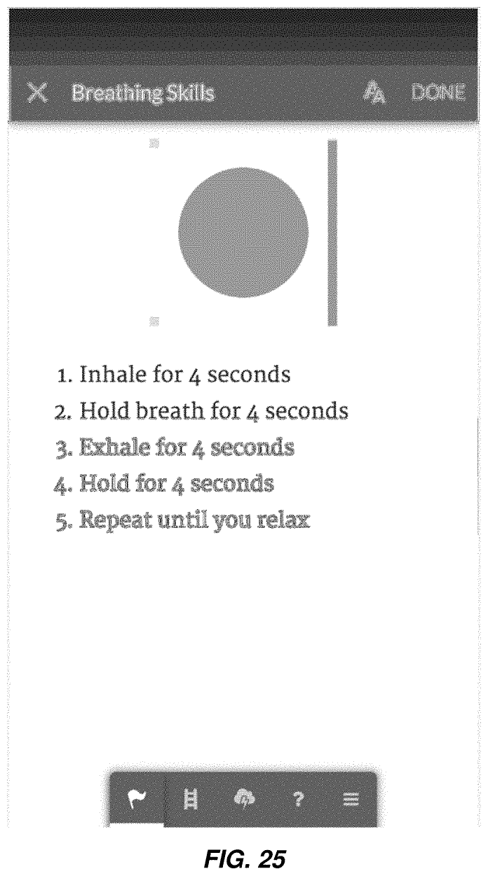

Responsive to selecting a trigger the application may display a list of suggested responses the user may undertake to mitigate or otherwise deal with the undesirable behavior or feeling. FIG. 20 illustrates a screen through which the user may select responses for a trigger. The interface of FIG. 20 is not yet populated with responses. The application allows the users to add alternative response to this trigger by clicking “Add Response” in the interface of FIG. 20. Responsive to the user clicking “Add Response” in the interface illustrated in FIG. 20, the application describes suggested responses for the selected trigger, as illustrated in FIG. 21. The application lists the most preferred suggested response at the top, but also lists other possible responses that are also acceptable. The user may click on a response in the interface illustrated in FIG. 21. Responsive to the user clicking on the response, the application opens a process for customizing the selected response for the selected trigger as illustrated in FIG. 22. When the user clicks add the application will add the selected response to the list of responses the user can try next time they encounter the selected trigger. FIG. 23 illustrates a populated version of the interface of FIG. 20 in which the application shows any responses that were previously selected by the user so that they may open the process associated with that response. FIGS. 24 and 25 illustrate screens that are displayed when the user opens the process associated response, engaging in the alternative behavior. These screens may guide the user in performing the response, for example, in performing breathing exercises as illustrated in FIG. 25.

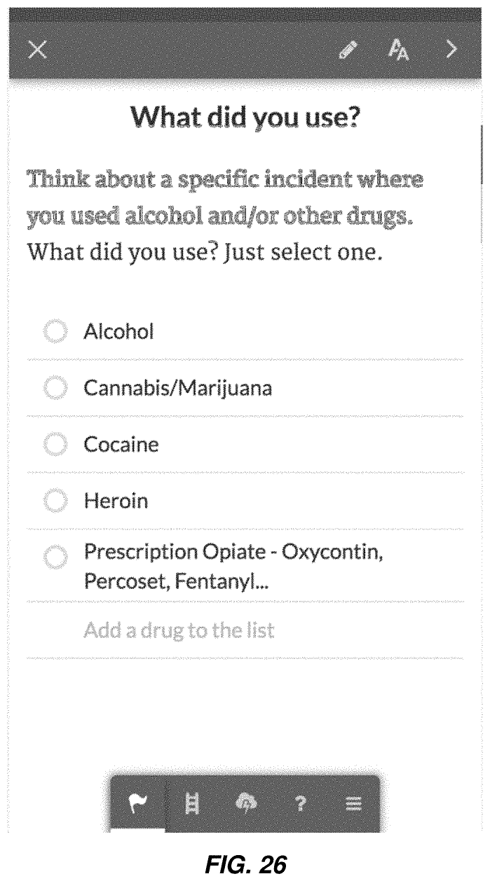

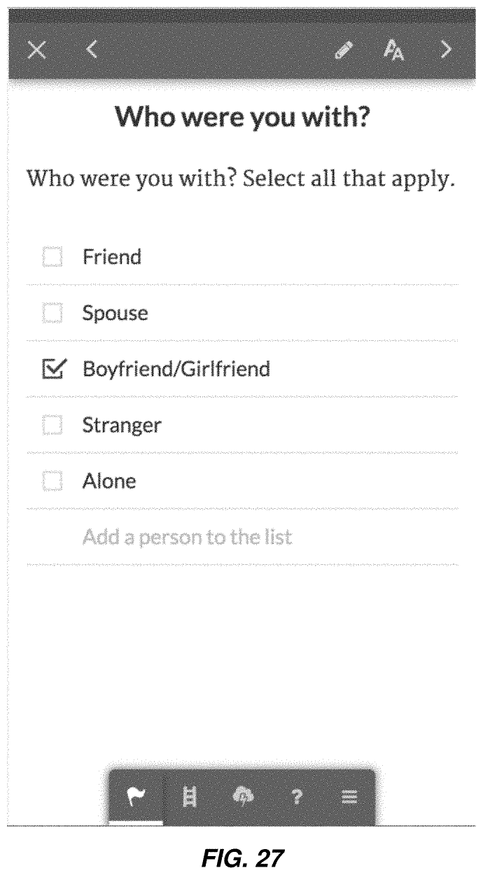

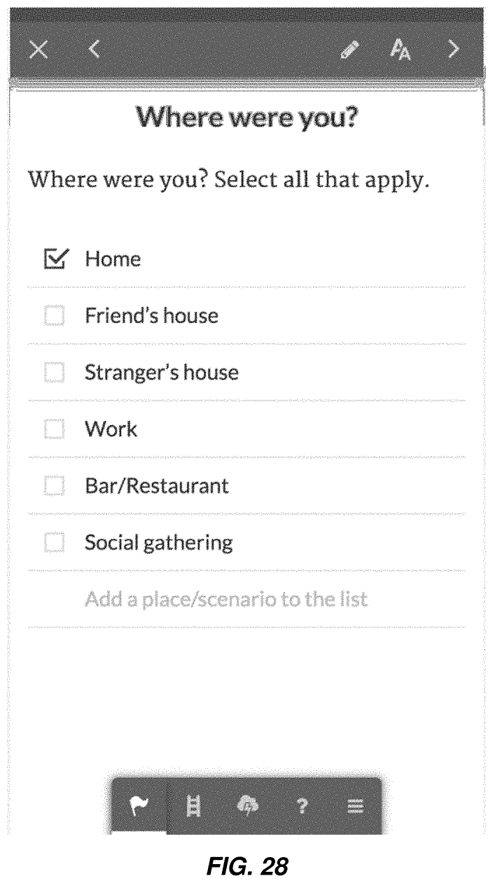

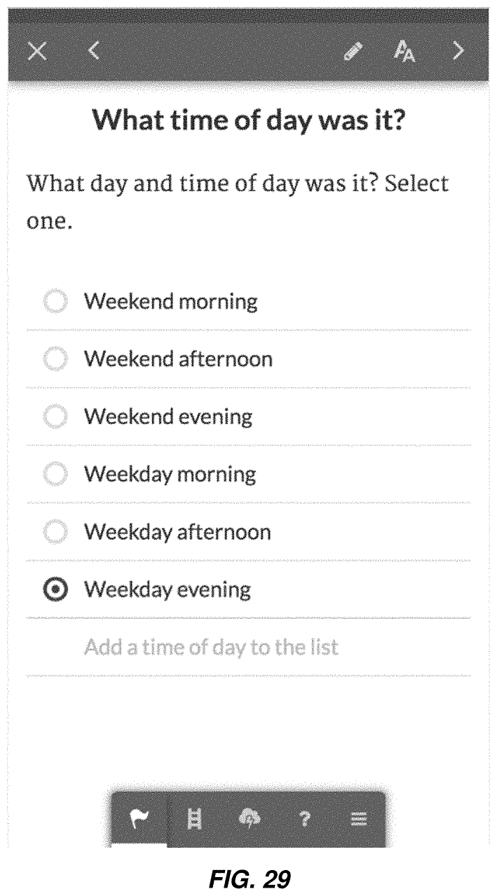

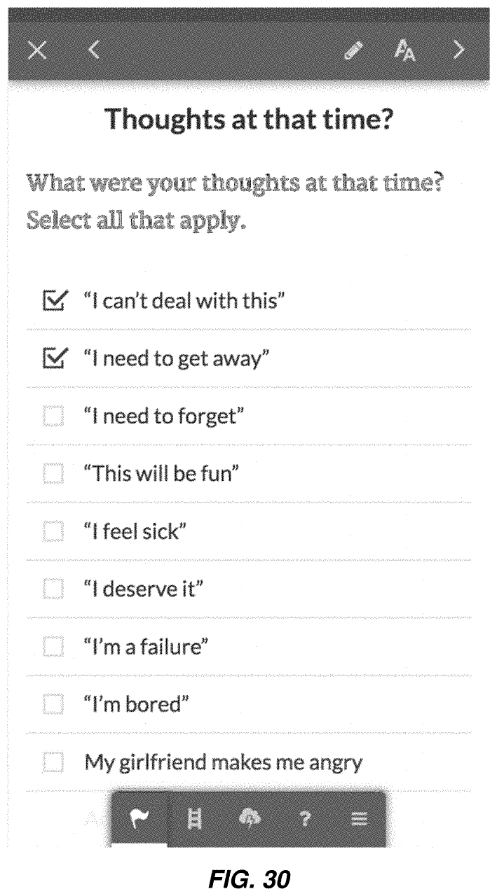

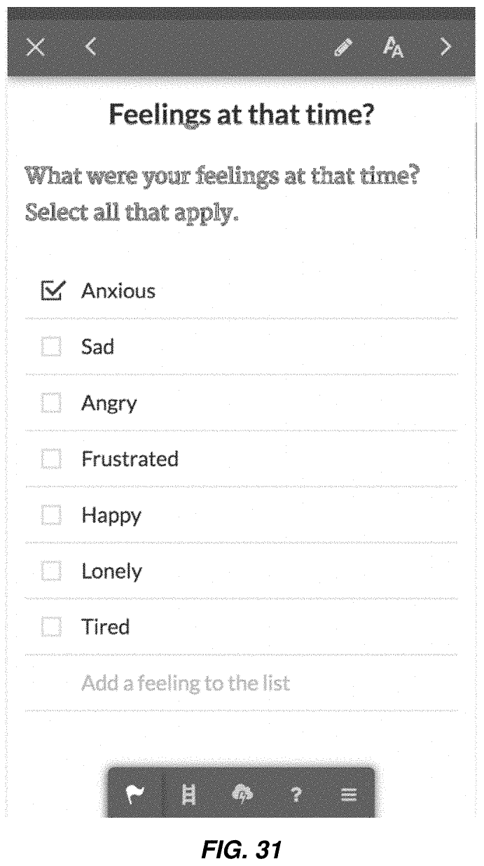

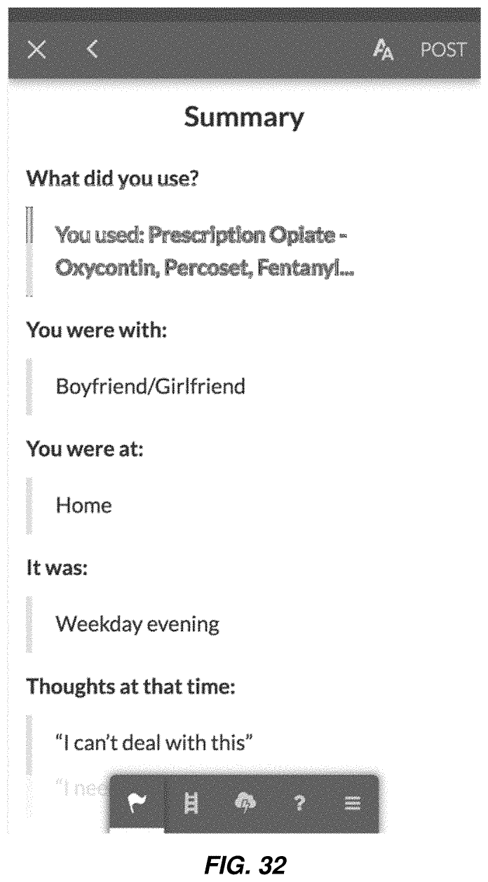

The application also provides for a user to identify triggers that may contribute to or cause the occurrence of an undesirable behavior or feeling. In an interface such as that illustrated in FIG. 26, the user identifies what behavior or feeing for which they are seeking to identify triggers. This can be free-text input or it may be built into the ladder to display a predetermined list of behaviors or feelings. In one embodiment the user is presented with a list of commonly used substances (alcohol, cannabis, cocaine, heroin, other opioids, etc.) The user may enter a substance not included in the pre-populated list, for example, in the “Add a drug to the list” field illustrated in FIG. 26. The user is asked questions regarding external cues associated with the behavior or feeling, for example, the application may ask the user to answer the question “Who were you with?” when they engaged in the target behavior. The application presents a list of common answers gathered from research (FIG. 27). The user may enter an option not included in the pre-populated list, for example, in the “Add a person to the list” field illustrated in FIG. 27. The user is asked “Where were you?” when they engaged in the target behavior. The application presents a list of common answers gathered from research (FIG. 28). The user may enter an option not included in the pre-populated list, for example, in the “Add a place/scenario to the list” field illustrated in FIG. 28. The user is asked “What time of day was it?” when they engaged in the target behavior. The application presents a list of common answers gathered from research (FIG. 29). The user may enter an option not included in the pre-populated list, for example, in the “Add a time of day to the list” field illustrated in FIG. 29. The user is asked questions regarding internal cues associated with the behavior or feeling, for example, what feelings or thoughts they can remember having when they engaged in the target behavior. The application presents a list of common answers gathered from research (FIGS. 30, 31). The user may enter an option not included in the pre-populated lists, for example, in the “Add a feeling to the list” field illustrated in FIG. 31. The options not included in the pre-populated lists in any of the interfaces illustrated in any of FIGS. 26-31 may be entered by typing or by utilizing speech-to-text voice recognition functionality of a device upon which embodiments of the software app disclosed herein may be installed. The user is presented with a summary of their responses (FIG. 32). These responses are used in a later Trigger Mapping process, for example, to generate a tallied list of cues or triggers associated with the behavior or feeling as illustrated in FIG. 19.

The app described above may be utilized as a stand-alone tool for a user to help a user achieve their goals. In other embodiments, information entered into the app may be shared, for example with a therapist and the therapist may use the data to make suggestion to the user to help thee user achieve their goals or to initiate an intervention if necessary.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled,” as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

Directional terms such as “above,” below,” “left,” “right,” etc. are used herein as a matter of convenience for referencing various surfaces and orientations of features disclosed herein. There directional terms do not imply that the aspects and embodiments disclosed herein are necessarily oriented in any particular orientation. Any dimensions provided in the above disclosure are meant as examples only and are not intended to be limiting.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while acts of the disclosed processes are presented in a given order, alternative embodiments may perform routines having acts performed in a different order, and some processes or acts may be deleted, moved, added, subdivided, combined, and/or modified. Each of these processes or acts may be implemented in a variety of different ways. Also, while processes or acts are at times shown as being performed in series, these processes or acts may instead be performed in parallel, or may be performed at different times.

The teachings of the invention provided herein can be applied to other systems, not necessarily the system described above. The elements and acts of the various embodiments described above can be combined to provide further embodiments.

Having thus described several aspects of at least one embodiment of this invention, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure, and are intended to be within the scope of the invention. Accordingly, the foregoing description and drawings are by way of example only.

1-75. (canceled)

78-85. (canceled)

86. An electronic device including a software application for assisting a user to achieve a behavioral modification goal, the application including programming that causes the device to display a user interface defining a ladder including a plurality of rungs, each of the rungs having one or more associated tasks, completion of the one or more associated tasks by the user causing the associated rung to be indicated as completed, at least one upper rung in the ladder requiring a lower rung in the ladder to be completed prior to unlocking the at least one upper rung, at least one rung in the ladder requiring multiple different chains to be completed for the rung to be unlocked.

87. The electronic device of claim 86, wherein the software application provides for a user to concurrently work at least two of the plurality of rungs.

88. The electronic device of claim 86, wherein the software application is programmed to display a ladder selection screen providing for the user to select a ladder designed to address behavior associated with a particular type of issue that the user exhibits, ladders designed to address behavior associated with particular types of issues including predefined rungs and associated tasks.

89. The electronic device of claim 88, wherein the ladder selection screen includes an option for the user to create a personalized ladder.

90. The electronic device of claim 86, wherein the software application is further programmed to display a user interface including a grid summarizing scores of a plurality of measures associated with different goals selected by the user and an axis including dates associated with respective scores of the plurality of measures.

91. The electronic device of claim 86, wherein the software application is programmed to identify positive or negative trend reversals in measures and direct the user to an appropriate intervention based on a direction of the trend reversal, measure data, and other user data.

92. The electronic device of claim 86, including functionality for tracking performance of a repeatedly performed activity and for initiating a rescue intervention responsive to the user failing to perform the repeatedly performed activity a threshold number of times within a defined time interval.

93. The electronic device of claim 92, wherein the rescue intervention includes a questionnaire configured to assist the user in identifying a reason for failing to perform the repeatedly performed activity the threshold number of times within the defined time interval.

94. The electronic device of claim 86, wherein responsive to the user indicating an occurrence of an undesired behavior in an interface of the electronic device, the software application presents a second user interface including a plurality of triggers from which the user may select a trigger that the user believed contributed to the occurrence of the undesired behavior.

95. The electronic device of claim 94, wherein the software application provides for the user to add their own triggers the second user interface via one of free-text input and voice recognition.

96. The electronic device of claim 94, wherein responsive to a selection of a trigger by the user, the software application presents the user with a list of one or more alternative behaviors based on one of the selected trigger and a category of the selected trigger, the alternative behaviors selected based on an expected efficacy of the alternative behaviors in alleviating an undesirable response of the user to one of the selected trigger or the category of the selected trigger.

97. The electronic device of claim 86, wherein one or more of the different chains are user definable and the application is programmed to cause the electronic device to display a second user interface including one or more features for assisting the user in defining the one or more of the different chains.

98. The electronic device of claim 86, wherein at least one rung on the ladder requires a user to achieve a score of a measure indicative of achievement of a user-defined outcome for the at least one rung to be unlocked.

99. The electronic device of claim 86, including functionality to utilize motivational interviewing techniques to re-engage the user after a period of non-use of the software application.

100. The electronic device of claim 99, configured to initiate notification of the user via one or more of SMS, email, or device notification after a period of non-use of the software application.

101. The electronic device of claim 100, wherein activation of the notification by the user opens the software application and engages the user in a process to motivate the user to continue to use the software application.

102. A device that facilitates the definition of a behavior modification goal by a user, applies behavioral modification techniques to assist the user in achievement of the behavior modification goal, and tracks progress toward achievement of the behavior modification goal by the user, the device including:
programming that causes the device to display a user interface that provides the user with information regarding progress toward achievement of the behavior modification goal; and
programming that causes the device to display a selection interface providing the user with a list of user-selectable behaviors or feelings that the user engaged in or experienced, and that causes the device to display one or more user-selectable lists of one or both of external or internal cues associated with a behavior or feeling selected by the user in the selection interface responsive to selection of the behavior or feeling by the user in the selection interface, and, responsive to the user selecting one of the external or internal cues from the one or more lists, to display a list of suggested responses the user may undertake to mitigate the behavior or feeling.

103. The device of claim 102, wherein the programming is further configured to prompt the user to enter one or both of an external or internal cue associated with the behavior or feeling selected by the user in the selection interface in a free-form text entry field responsive to selection of the behavior or feeling by the user in the selection interface or responsive to the user.

104. The device of claim 102, wherein the programming is further configured to guide the user through performance of a response that the user selects from the list of suggested responses.

105. The device of claim 102, wherein the programming further causes the device to present a user interface defining a ladder including a plurality of rungs, each of the rungs having one or more associated tasks, completion of the one or more associated tasks by the user causing the associated rung to be indicated as completed, at least one upper rung in the ladder requiring a lower rung in the ladder to be completed prior to unlocking the at least one upper rung, at least one rung in the ladder requiring multiple different chains to be completed for the rung to be unlocked.
